(19) United States
(12) Patent Application Publication
(10) Pub. No.: US 2023/0240611 A1
(43) Pub. Date: Aug. 3, 2023

(54) IN-EAR SENSORS AND METHODS OF USE THEREOF FOR AR/VR APPLICATIONS AND DEVICES

(71) Applicant: Meta Platforms Technologies, LLC, Menlo Park, CA (US)

(72) Inventors: Morteza Khaleghimeybodi, Bothell, WA (US); Barry David Silverstein, Kirkland, WA (US); Nils Thomas Fritiof Lunner, Redmond, WA (US); John Rumsfeld, San Francisco, CA (US); Andrew John Ouderkirk, Kirkland, WA (US); Joshua Weiner, Great Neck, NY (US)

(21) Appl. No.: 18/069,106

(22) Filed: Dec. 20, 2022

Related U.S. Application Data
(60) Provisional application No. 63/305,932, filed on Feb. 2, 2022.
(60) Provisional application No. 63/356,851, filed on Jun. 29, 2022.
(60) Provisional application No. 63/356,860, filed on Jun. 29, 2022.
(60) Provisional application No. 63/356,864, filed on Jun. 29, 2022.
(60) Provisional application No. 63/356,872, filed on Jun. 29, 2022.
(60) Provisional application No. 63/356,877, filed on Jun. 29, 2022.
(60) Provisional application No. 63/356,883, filed on Jun. 29, 2022.

(51) Int. Cl. A61B 5/00; H04R 1/10; A61B 5/0205

(52) U.S. Cl. CPC A61B 5/6817; H04R 1/1016; A61B 5/7214; A61B 5/0205; A61B 5/282

(57) ABSTRACT

A computer method for managing, processing and handling sensor signals from an in-ear monitor for immersive reality applications is provided. The method includes receiving, from a first electrode, a first electronic signal from a skin in a first ear canal of a user of an in-ear device, receiving, from a first microphone, a first acoustic signal from the first ear canal of the user of the in-ear monitor, and a second acoustic signal from a second ear canal of the user (binaural data capture), forming an acoustic waveform with the first acoustic signal, forming an electronic waveform with the first electronic signal, and identifying a health condition of the user based on the acoustic waveform and the electronic waveform. A device including a memory storing instructions and processors to execute the instructions to perform the above method are also provided.

### CROSS-REFERENCE TO RELATED APPLICATIONS

The present disclosure is related and claims priority under 35 U.S.C. § 119(e) to U.S. Prov. Appln. No. 63/305,932, entitled IN-EAR BIO-SENSING FOR AR/VR APPLICATIONS AND DEVICES, filed on Feb. 2, 2022, to U.S. Prov. Appln. No. 63/356,851, entitled IN-EAR ELECTRODES FOR AR/VR APPLICATIONS AND DEVICES, to U.S. Prov. Appln. No. 63/356,860, entitled IN-EAR OPTICAL SENSORS FOR AR/VR APPLICATIONS AND DEVICES, to U.S. Prov. Appln. No. 63/356,864, entitled IN-EAR MOTION SENSORS FOR AR/VR APPLICATIONS AND DEVICES, to U.S. Prov. Appln. No. 63/356,872, entitled IN-EAR TEMPERATURE SENSORS FOR AR/VR APPLICATIONS AND DEVICES, to U.S. Prov. Appln. No. 63/356,877, entitled IN-EAR MICROPHONES FOR AR/VR APPLICATIONS AND DEVICES, to U.S. Prov. Appln. No. 63/356,883, entitled IN-EAR SENSORS AND METHODS OF USE THEREOF FOR AR/VR APPLICATIONS AND DEVICES, all filed on Jun. 29, 2022, to Morteza KHALEGHIMEYBODI, et al., the contents of which applications are hereby incorporated by reference in their entirety, for all purposes.

### BACKGROUND

### Field

The present disclosure is related to in-ear sensors for use in virtual reality and augmented reality environments and devices. More specifically, the present disclosure is related to sensors configured to receive input from the inside and outside the ear for health monitoring with in-ear devices for immersive reality applications.

### Related Art

Current in-ear devices (e.g., hearing aids, hearables, headphones, earbuds, and the like) for mobile and immersive applications are typically bulky and uncomfortable for the user. Adding health sensing capabilities to in-ear devices is hindered by the small form factors desirable in such devices and the complex data processing and analysis involved.

### SUMMARY

In a first embodiment, a computer-implemented method includes receiving, from a first electrode, a first electronic signal from a skin in a first ear canal of a user of an in-ear device, receiving, from a first microphone, a first acoustic signal from the first ear canal of the user of the in-ear monitor, forming an acoustic waveform with the first acoustic signal, forming an electronic waveform with the first electronic signal, and identifying a health condition of the user based on the acoustic waveform and the electronic waveform.

In a second embodiment, a computer-implemented method includes receiving, from a first electrode, a first electronic signal from a skin in a first ear canal of a user of an in-ear device, transmitting, into the ear canal of the user of the in-ear device, a first electromagnetic radiation, receiving, from an electromagnetic detector, a signal indicative of a second electromagnetic radiation responsive to the first electromagnetic radiation, forming an electronic waveform with the first electronic signal, forming an electromagnetic waveform based on the first electromagnetic radiation and the second electromagnetic radiation, and identifying a health condition of the user based on the electronic waveform and the electromagnetic waveform.

In a third embodiment, a computer-implemented method includes receiving a first waveform indicative of one of an electrical activity from a body of a user of an in-ear monitor, receiving a second waveform indicative of one of an acoustic signal, an inner motion of the user of the in-ear monitor, or a bulk motion of the user of the in-ear monitor, generating an array of values, wherein each value includes portions of the first waveform and the second waveform weighted by a coefficient, identifying a condition of the user from the array of values, determining a loss value based on a comparison between the condition of the user and a ground truth condition, and updating at least one of the coefficients when the loss value is larger than a pre-selected threshold.

In a fourth embodiment, a computer-implemented method includes receiving, from a sensor in an in-ear device, a signal indicative of a vital sign of a user of the in-ear device, updating a timeline of a medical condition of the user of the in-ear device with the signal indicative of the vital sign from the user, determining a trend for the medical condition of the user based on the timeline of the medical condition, identifying one or more deviations in the trend for the medical condition relative to a reference trendline, and projecting a medical outcome for the user of the in-ear device based on the one or more deviations.

In other embodiments, a non-transitory, computer-readable medium stores instructions which, when executed by a processor, cause a computer to perform a method. The method includes, receiving, from a first electrode, a first electronic signal from a skin in a first ear canal of a user of an in-ear device, receiving, from a first microphone, a first acoustic signal from the first ear canal of the user of the in-ear monitor, forming an acoustic waveform with the first acoustic signal, forming an electronic waveform with the first electronic signal, and identifying a health condition of the user based on the acoustic waveform and the electronic waveform.

In yet other embodiments, a system includes a first means to store instructions, and a second means to execute the instructions to cause the system to perform a method. The method includes, receiving, from a first electrode, a first electronic signal from a skin in a first ear canal of a user of an in-ear device, receiving, from a first microphone, a first acoustic signal from the first ear canal of the user of the in-ear monitor, forming an acoustic waveform with the first acoustic signal, forming an electronic waveform with the first electronic signal, and identifying a health condition of the user based on the acoustic waveform and the electronic waveform.

These and other embodiments will become clear to one of ordinary skill in the art in view of the following.

### BRIEF DESCRIPTION OF THE FIGURES

FIG. 1 illustrates an AR headset and an in-ear monitor (IEM) in an architecture configured to assess a user's health, according to some embodiments.

FIG. 2 illustrates an augmented reality ecosystem including wearable devices in the ear and wrist to assess a user's health, according to some embodiments.

FIGS. 3A-3D illustrate different embodiments of an in-ear monitor (IEM), according to some embodiments.

FIG. 4 illustrates an IEM signal combined with an ECG signal, and a blood pressure regression chart, according to some embodiments.

FIG. 5 illustrates a waveform obtained with a contact microphone in an IEM to determine a heart rate of a user, according to some embodiments.

FIG. 6 illustrates a combination of an ear PPG waveform, an ECG waveform, and a wrist PPG waveform for identifying a health condition of a user of an in-ear device, according to some embodiments.

FIG. 7 illustrates several regression methods to combine features in the waveforms of FIG. 6 to identify a health condition of a user of an in-ear device, according to some embodiments.

FIG. 8 is a flow chart illustrating steps in a method for using microphones, motion sensors, optical sensors, electrodes, and a thermometer in an in-ear monitor for assessing the health of a user of a headset or smart glass, according to some embodiments.

FIG. 9 is a flow chart illustrating steps in a method for using microphones, motion sensors, optical sensors, electrodes, and a thermometer in an in-ear monitor for assessing the health of a user of a headset or smart glass, according to some embodiments.

FIG. 10 is a flow chart illustrating steps in a method for using microphones, motion sensors, optical sensors, electrodes, and a thermometer in an in-ear monitor for assessing the health of a user of a headset or smart glass, according to some embodiments.

FIG. 11 is a flow chart illustrating steps in a method for creating a data of measurements over time to create a reference and trendline, according to some embodiments.

FIG. 12 is a block diagram illustrating an exemplary computer system with which headsets and other client devices, and the methods in FIGS. 8-11 be implemented, according to some embodiments.

In the figures, elements having the same or similar reference numeral are associated with the same or similar attribute, unless explicitly expressed otherwise.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

General Overview

Head-worn devices (e.g., devices worn on head including but not limited to hearables, smart glasses, AR/VR headsets and smart glasses, etc.) offer opportunities to access valuable health information.

The ear (e.g., the ear canal and ear concha and pinna) has close proximity to the brain, to body chemistry, and blood vessels indicative of brain activity and cardio-respiratory activity, and inner body temperature. More specifically, sensors including electrodes, inertial motion units (IMUs), accelerometers, and microphones can be placed inside the ear canal or around the ear (in the case of AR/VR headsets or smart glasses) to sense brain, heart, and ocular electrophysiological activities (e.g., electro-encephalography, EEG, electro-cardiography, ECG, electro-oculography, EOG, electrodermal activity, EDA, and the like); or to sense vital signs (heart rate, breathing rates, blood pressure, body temperature, and the like); or to sense the body chemistry (e.g., blood alcohol level, blood glucose estimation, and the like).

Embodiments as disclosed herein provide temperature measurements in the ear are attractive because the ear is close to the inner body temperature. For example, an artery going through the eardrum feeds directly through the hypothalamus, which is the part of the brain that controls body temperature. Thus, this can provide more accurate continuous core body temperature sensing for the user compared to having temperature sensors at other body locations such as wrist and finger. In addition, the high density of blood vessels and nerve structures in the ear canal make it an information-rich area that is convenient to access from the point of view of continuous and unobtrusive monitoring and measurement.

Some embodiments include microphones such as contact microphones to detect motion, internal microphones and external microphones, acoustic microphones, and the like. In addition to microphones, in-ear devices as disclosed herein may also include speakers to generate and provide sound signals to the user of the in-ear device.

Electrodes in embodiments as disclosed herein may be used in EOG, ECG, and EEG measurements, e.g., for determining auditory attention; heart rate estimation, breathing rate, and the like, Auditory Steady State Response—ASSR—, auditory brainstem response—ABR—. In some embodiments, in-ear electrodes as disclosed herein may be useful to measure resting state electric oscillations (alpha waves in an EEG) that can track relaxation/activity. With the combination of other measurements (e.g., photoplethysmography, PPG), a new branch of diagnostic possibilities is open. In-ear EEG measurements can be applied to track user attention (e.g., distinguishing between attention focus from eye gaze direction).

Methods and devices disclosed herein include motion, optical, acoustical, motion sensors, chemical sensors, and temperature sensors, in and around the ears of AR/VR headset users, in combination with software correlation of the signals provided by the above sensors to generate comprehensive diagnostics and health evaluation of the user.

Some of the features disclosed herein include in-ear or head-worn body temperature sensing using infrared sensing and spectroscopy techniques. In some embodiments, the contact area for sensors as disclosed herein include the in-ear canal (like an in-ear earbud) and within the conchal bowl (in human pinna), areas on top of the human ear (where the glasses sit), and areas in the nose-pad of a headset or smart glass (where glasses sit on the nose). Some measurements may include in-ear or around the ear sensing of glucose level, alcohol sensing, body temperature, blood pressure, and the like. Some embodiments include pulse transit time (PTT) methodology to estimate blood pressure for a glass/headset device using a combination of optical and electrical signals (e.g., PPG+ECG sensors respectively) or using a combination of electrical and acoustical or motion-based information (e.g., ECG+acoustic or motion sensors respectively). Some embodiments include optical-based pulse transit time (PTT) methodology to estimate blood pressure for a glasses/headset device using a combination of optical signals collected from multiple different wavelength (e.g., using a PPG sensor with more than one distinct wavelength). Some embodiments obtain user's blood pressure using an optical sensing technique (PPG) in combination with a deep neural network to train a network based using both PPG information and a corresponding ground-truth blood pressure information. Some embodiments include motion-based pulse transit time (PTT) methodology to estimate blood pressure for a glass/headset device using a combination of motion sensor and electrical signals (e.g., IMU+ECG sensors respectively). Once fully trained, the neural network can then quantify and predict the user's blood pressure using just the PPG information and leveraging this pre-trained network. To further improve the accuracy, some subjective calibrations may be desirable. In some embodiments, PPG signals collected in IEM devices as disclosed herein may be able to estimate the cognitive load on the user with analysis of oxygenated and deoxygenated blood flow (oxy- and deoxy-hemoglobin) to the brain. Some embodiments include sensing alcohol levels through emissions around the ear. Some embodiments incorporate chemical sensing intake around the contact points of the ear. In some embodiments, IEM devices may perform alcohol monitoring and fat burning during user exercise.

### Example System Architecture

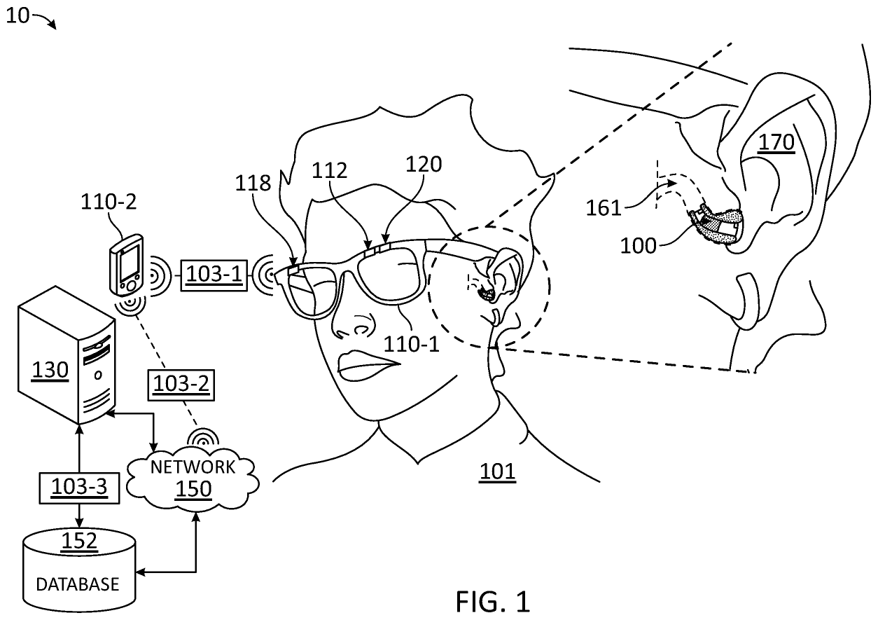

FIG. 1 illustrates an AR headset 110-1 and an in-ear monitor (IEM) 100 in an architecture 10 configured to assess the health of a user 101, according to some embodiments. IEM 100 is inserted in the ear 170 of user 101, reaching the ear canal 161. AR headset 110-1 may include smart glasses having a memory circuit 120 storing instructions and a processor circuit 112 configured to execute the instructions to perform steps as in methods disclosed herein. AR headset 110-1 (or smart glasses) may also include a communications module 118 configured to wirelessly transmit information (e.g., Dataset 103-1) between AR headset 110-1 (and/or in-ear device 100, and/or a smart watch, or combination of the above) and a mobile device 110-2 with the user (AR headset 110-1 and mobile device 110-2 will be collectively referred to, hereinafter, as “client devices 110”). Communications module 118 may be configured to interface with a network 150 to send and receive information, such as dataset 103-1, dataset 103-2, and dataset 103-3, requests, responses, and commands to other devices on network 150. In some embodiments, communications module 118 can include, for example, modems or Ethernet cards. Client devices 110 may in turn be communicatively coupled with a remote server 130 and a database 152, through network 150, and transmit/share information, files, and the like with one another (e.g., dataset 103-2 and dataset 103-3). Datasets 103-1, 103-2, and 103-3 will be collectively referred to, hereinafter, as “datasets 103.” Network 150 may include, for example, any one or more of a local area network (LAN), a wide area network (WAN), the Internet, and the like. Further, the network can include, but is not limited to, any one or more of the following network topologies, including a bus network, a star network, a ring network, a mesh network, a star-bus network, tree or hierarchical network, and the like.

In some embodiments, at least one of the steps in methods as disclosed herein are performed by processor 112, providing dataset 103-1 to mobile device 110-2. Mobile device 110-2 may further process the signals and provide dataset 103-2 to database 152 via network 150. Remote server 130 may collect dataset 103-2 from multiple AR headsets 110-1 and mobile devices 110-2 in the form and perform further calculations. In addition, having aggregated data from a population of individuals, the remote server may perform meaningful statistics. This data cycle may be established provided each of the users involved have consented for the use of de-personalized, or anonymized data. In some embodiments, remote server 130 and database 152 may be hosted by a healthcare network, or a healthcare facility or institution (e.g., hospital, university, government institution, clinic, health insurance network, and the like). Mobile device 110-2, AR headset 110-1, in-ear device 100, and applications therein may be hosted by a different service provider (e.g., a network carrier, an application developer, and the like). Moreover, AR headset 110-1 and mobile devices 110-2 may proceed from different manufacturers. User 101 is ultimately the sole owner of dataset 103-1 and all data derived therefrom (e.g., datasets 103), and so all the data flows (e.g., datasets 103), while provided, handled, or regulated by different entities, are authorized by user 101, and protected by network 150, server 130, database 152, and mobile device 110-2 for privacy and security.

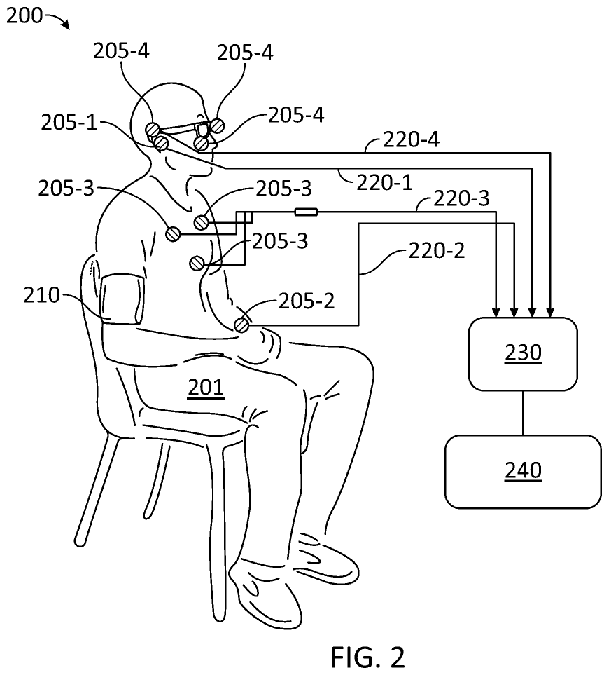

FIG. 2 illustrates an augmented reality ecosystem 200 including wearable devices in the ear 205-1 (e.g., an IEM), wrist 205-2, chest 205-3, and smart glass sensors 205-4 to assess the health of user 201, according to some embodiments. In some embodiments, IEM 205-1 further includes an optical sensor configured to provide an optical signal 220-1 to a processor in a computer 240 via a data acquisition module (DAQ) 230. IEM 205-1 may further include one or more contact electrodes configured to provide an electrical signal to a processor in a computer 240 via a data acquisition module (DAQ) 230. Computer 240 is configured to identify a cardiovascular condition of user 201 based on a first electronic signal from IEM 205-1 and optical signal 220-1. In some embodiments, IEM 205-1 further includes a motion sensor (e.g., an accelerometer, a contact microphone, or an IMU) configured to provide a motion-based signal to computer 240 via DAQ 230. In some embodiments, a pair of IEMs 205 will be placed in both ears and different optical, electrical (electrode), acoustic (microphone), or motion sensors (accelerometer, IMU, contact microphone, etc.) may be placed in either both sides; or in some cases, some sensors may be placed on one side (e.g., the Right side) and some other sensors may be placed on the other side (e.g., Left side). Computer 240 is configured to identify a cardiovascular condition of the user based on a first electronic signal from IEM 205-1 and the motion signal. The optical sensor may be a photo-plethysmography (PPG) sensor and optical signal 220-1 may include a digital or analog signal indicative of a vascular activity inside the ear of user 201. Chest sensors 205-3 and smart glass sensors 205-4 may include ECG sensors to provide a distributed signals 220-3 and 220-4 from one or more areas around the chest and face (e.g., the outside of the ear, the chin, and the nose) of user 201, respectively (or alternatively an ECG can be collected from some electrodes placed on areas on the head or from electrodes placed in IEM 205-1, or electrodes placed on the wrist device 205-2), and a wrist PPG sensor in device 205-2 may provide a separate signal 220-2 for vascular activity around the wrist of user 201. IEM 205-1, wrist sensor 205-2, chest sensors 205-3, and smart glass sensors 205-4 will be collectively referred to, hereinafter, as “wearable devices (and sensors) 205.” Blood pressure (BP) measurements may be obtained with a cuff or cuff-less BP monitor 210 and may also be determined by comparing PPG signals 220-1 and 220-2. Signals 220-1, 220-2, 220-3 and 220-4 (hereinafter, collectively referred to, hereinafter, as “signals 220”) may be collected and digitized by DAQ 230 in computer 240, for processing. In some embodiments, signals 220 and others may be wired, or wireless. In some embodiments, it may be preferable to have wireless signal communication between the different wearable devices 205 with user 201. In some embodiments, wearable devices and sensors 205 may include one or more motion sensors, and the motion-based information collected from the smart glass, the IEM, chest or wrist can be combined to create a more meaningful information.

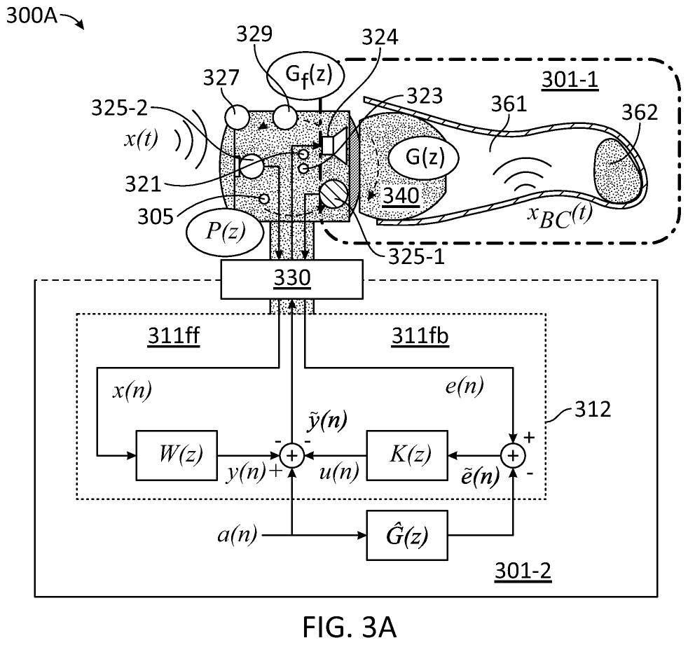

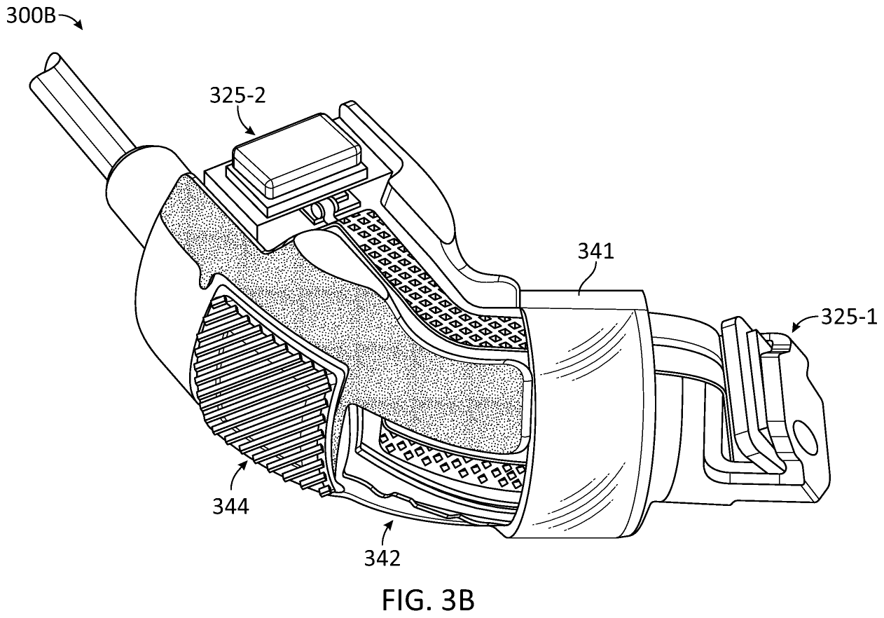

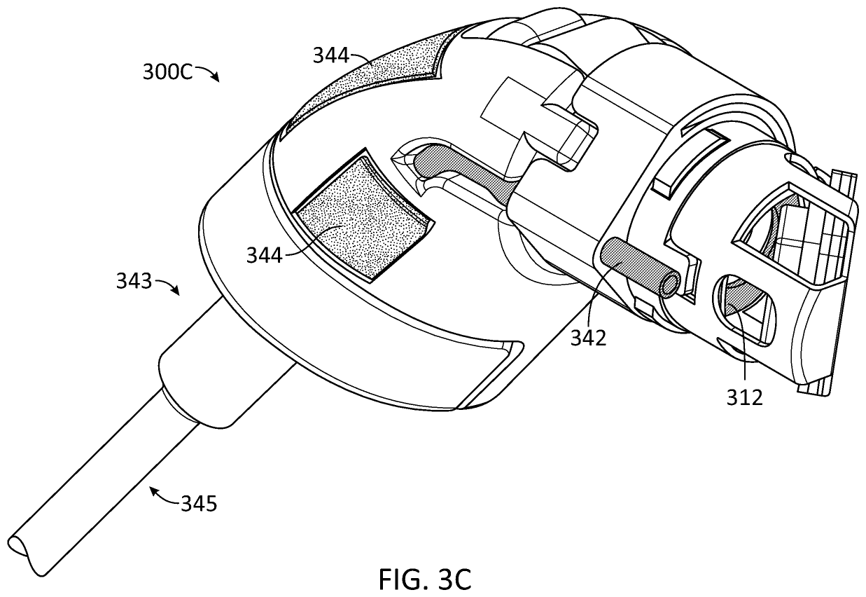

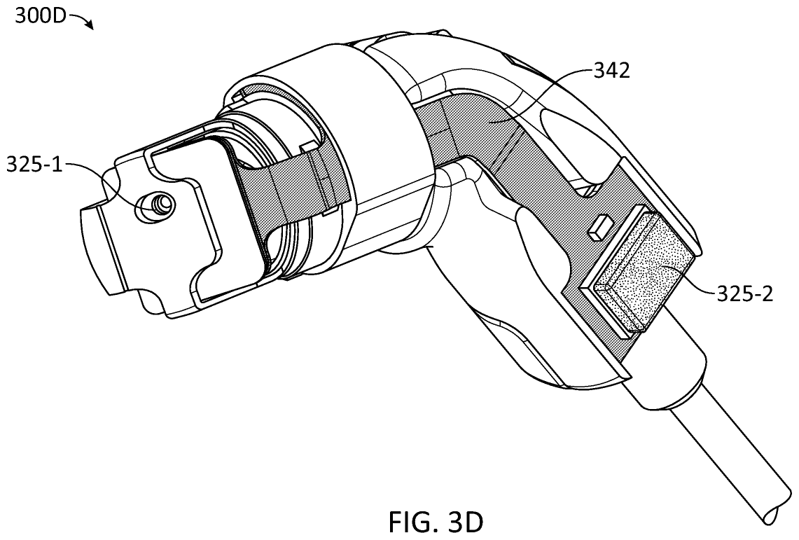

FIGS. 3A-3D illustrate different embodiments of an in-ear monitor (IEM) 300A, 300B, 300C, and 300D (hereinafter, collectively referred to as “IEMs 300”), according to some embodiments. IEMs 300 may include a front end 301-1 including sensors and open to ear canal 361 and ear drum 362, and a back end 301-2 including a processor 312. IEMs 300 may include sensors such as: an electrode 305 to sense electrical signals, acoustic sensors 325-1 and 325-2 (e.g., collectively referred hereinafter, as “microphones 325”), motion sensors 327 (e.g., accelerometers, contact microphones, inertial motion units—IMUs, and the like), temperature sensors 329, and optical sensors including an emitter 321 and a detector 323 (e.g., LEDs and PDs in PPG sensors, functional near-infrared spectroscopy fNIRS sensors—Fourier transform based, spectroscopic based—). Electrodes 305 may include bio-potential electrodes for applications such as EEG, ECG, EOG, and EDA). In some cases, the in-ear fixture 340 (also known as eartip) may be entirely made out of soft conductive materials; so, the entire eartip will be conductive and will act as a soft electrode. In addition, processor 312 may handle at least some of the operations for signal acquisition and control of components and sensors 321, 323, 324 (a speaker), 325-1 (internal microphone), 325-2 (external microphone, hereinafter, collectively referred to as “microphones 325”), 327, and 329 via a digital-to-analog and/or analog-to-digital converter (DAC/ADC) 330. Processor 312 may include a feedforward stage 311ff and a feedback stage 311fb that cooperate to process the signal from the sensors: noise reduction, balancing, filtering, and amplification.

In some embodiments, electrodes 305 include a contact electrode configured to transmit a current from the skin in the ear canal of the user. In some embodiments, an electrode 305 is coated with at least one of a gold layer, a silver layer, a silver chloride layer, or a combination thereof. In some embodiments, electrodes 305 include a capacitive coupling electrode disposed sufficiently close, but not in contact, with the user's skin. In some embodiments, IEMs 300 further include at least a second electrode 305 mounted on in-ear fixture 340, the second electrode 305 configured to receive a second electronic signal from the skin in ear canal 361. In some embodiments, in-ear fixture 340 may be entirely made out of soft conductive materials (e.g., conductive polymers, conductive adhesives, conductive paints, etc.); so, the entire eartip will be conductive and will act as a soft electrode to collect electrical signals from the skin of the ear-canal. In some embodiments, processor 312 is configured to select the first electronic signal when a quality of the first electronic signal is higher than a pre-selected threshold. In some embodiments, processor 312 is configured to reduce a noise background from the first electronic signal with the second electronic signal. In some embodiments, processor 312 is configured to determine a heart rate of the user from the first electronic signal. In some embodiments, processor 312 is configured to determine a brain activity from the first electronic signal that corresponds to an acoustic stimulus received in the external microphone.

IEMs 300 in the AR headset or smart glasses may include an in-ear fixture 340 configured to hermetically seal an ear canal of a user, a first electrode 305 mounted on in-ear fixture 340 and configured to receive a first electronic signal from a skin in ear canal 361, and an internal microphone 325-1 coupled to receive an internal acoustic signal, propagating through ear canal 361. An acoustic front end includes internal microphone 325-1 configured to detect acoustic waves (x_{BC}(t)) propagated through ear canal 361 and generated by the inner body (e.g., heart rate at about <100 Hz, breathing rate at about 50-1000 Hz, and other sounds in the laryngeal cavity). An external microphone 325-2 is coupled to receive an external acoustic signal x(t), propagating through an environment of the user. In some embodiments, the internal signal x_{BC}(t) in conjunction with the external signal x(t) may be used in acoustic procedures such as audio streaming, hear-through, active noise cancelation (ANC), hearing corrections, virtual presence and spatial audio, call services, and the like. In some embodiments, at least some of the above processes are performed in conjunction between left-ear and right-ear IEM monitors 300.

In some embodiments, speaker 324 and internal microphone 325-1 may be part of a self-mixing interferometer (SMI). An SMI is a compact, low power, inexpensive and sensitive acoustic interferometry device configured to measure displacement of the skin based on acoustic interference patterns between a portion of an emitted acoustic wave and the acoustic wave reflected from the skin. In some embodiments, a displacement of the skin obtained with an SMI is combined with heart rate measurements (e.g., from PPG sensors, motion sensors or ECG electrodes) to measure blood pressure and heart rate, or even vibration of the eardrum to also act as an internal microphone.

IEM 300B includes a sealing gasket 341 that separates the inner portion of ear canal 361 from the environment, leaving a back-volume vent including an acoustically resistive mesh 344 for a pressure equalizer (PEQ) tube 342 to vent into resistive mesh 344 (also shown in IEM 300C). The sealed cavity may enable breathing and heart rate monitoring (e.g., isolating the signal from internal acoustic microphone 325-1) at low power usage and with a small form factor.

IEM 300C illustrates processor circuit 312 to identify a cardiovascular condition or a neurologic condition of the user, based on at least one of a first electronic signal, an internal acoustic signal, and an external acoustic signal (e.g., from microphones 325). Some embodiments may include a down cable 345 to electrically couple the IEM with the VR headset or smart glasses, including a strain relief 343.

IEM 300D illustrates a flexible, printed circuit board (FPCB) 342 that provides internal electrical connectivity to the different components and sensors 321, 323, 324, 325, 327, and 329.

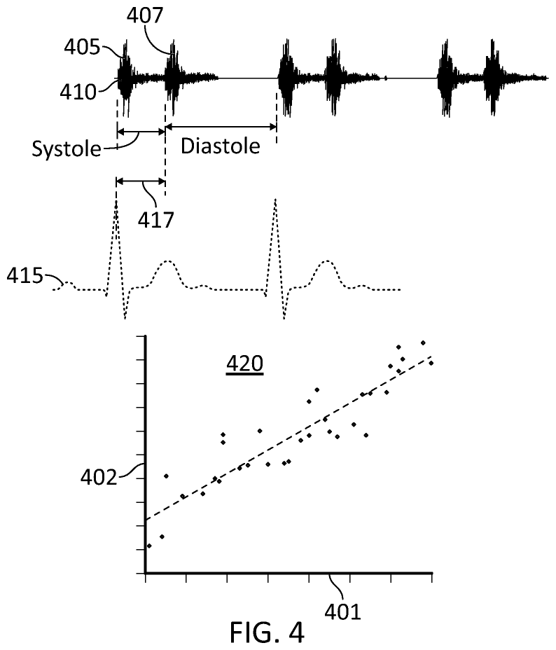

FIG. 4 illustrates an IEM signal 410 combined with an ECG signal 415, and a blood pressure regression chart 420, according to some embodiments. In some embodiments, an in-ear microphone signal forms an acoustic waveform 410 that may be overlapped with ECG signal 415 provided by an in-ear electrode (e.g., electrode 305), or any electrode disposed on a wearable device (e.g., a smart watch, or wristband 205-2, and the like). In some embodiments, a motion sensor signal from an IMU (e.g., accelerometer, gyroscope) may be used instead of IEM signal 410. ECG signal 415 provides a reference time for the start of a heart pulse, from which a systolic portion 405 and a diastolic portion 407 of acoustic waveform 410 of heart sounds may be identified. Accordingly, a time lapse 417 between the initial electronic pulse in ECG signal 415 and diastolic portion 407 may be indicative or have a direct correlation (e.g., a ratio 402 between systolic/diastolic portions 405 and 407) with a blood pressure 401 of the user. Other correlation factors to identify blood pressure 401 for the user may include a ratio between an amplitude of systolic portion 405 and diastolic portion 407.

In some embodiments, the spectral signature of systolic portion 405 and diastolic portion 407 may also be indicative of user's vital signs. It is generally observed that systolic portion 405 includes a narrower frequency bandwidth, while diastolic portion 407 has a broader bandwidth.

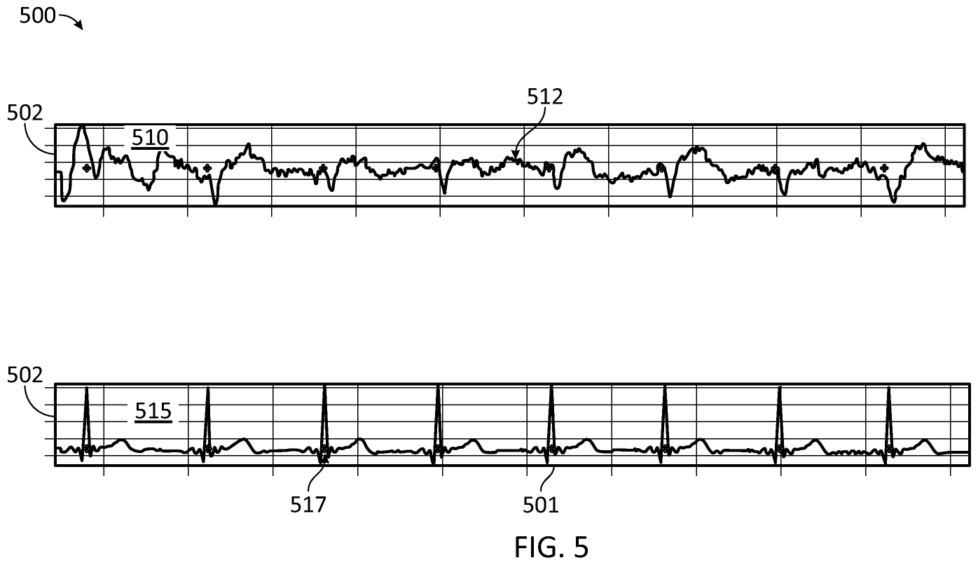

FIG. 5 is a chart 500 illustrating a waveform 510 obtained with a contact microphone in an IEM to determine a heart rate of a user, according to some embodiments. Chart 500 includes an abscissa 501 (e.g., time, in seconds), and an ordinate 502 (signal amplitude). Waveform 510 is obtained from a contact microphone inside the ear canal of an IEM user. In some embodiments, a similar waveform may be obtained with an IMU, accelerometer, and the like. A ground-truth ECG 515 includes the locations of R-peaks 517 of the user's heartbeat.

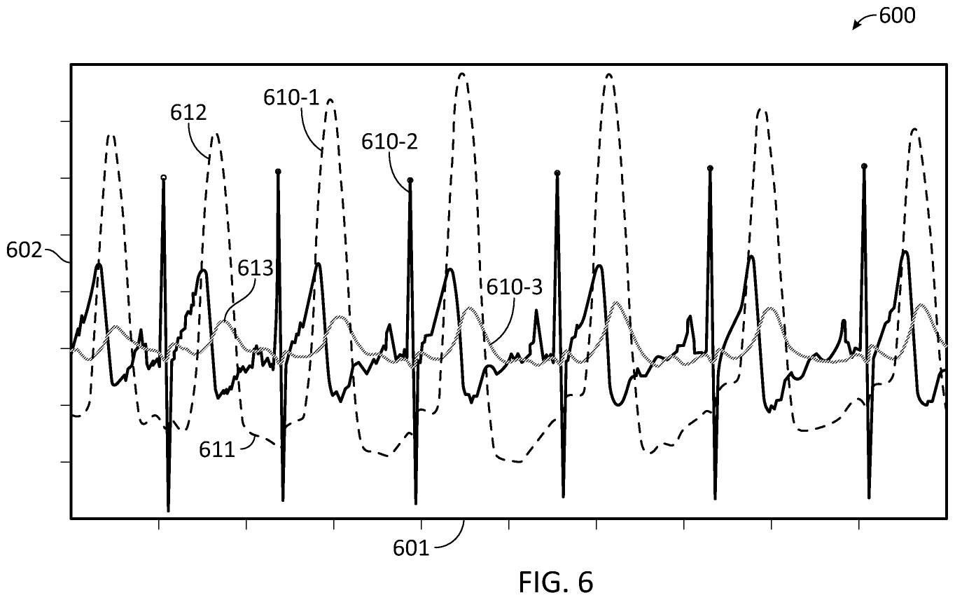

FIG. 6 illustrates a chart 600 with a combination of an ear PPG waveform 610-1, an ECG waveform 610-2, and a wrist PPG waveform 610-3 (hereinafter, collectively referred to as “waveforms 610”) for identifying a health condition of a user of an in-ear device, according to some embodiments. Chart 600 plots waveforms 610 with an abscissa 601 (e.g., time) an dan ordinate 602 (e.g., signal amplitude). It is observed that the peaks 612 of ear PPG waveform 610-1 occur earlier (˜30-40 msec) than the peaks 613 of wrist's PPG waveform 610-3 (the head is closer to the heart than the wrist). Another aspect of ear PPG waveform 610-1 is that the features are more pronounced compared to the wrist PPG waveform 610-3, as arteries in or around the ear are better perfused.

Other measurements are available from the waveform collected from a contact microphone. These include, in addition to heart rate and breathing rate, and without limitation: step count, pose estimation, and fall detection. Moreover, some embodiments enable blood pressure estimation using the pulse transit time (PTT) technique combining a contact microphone/motion sensor and an ECG sensor.

A time lapse between Ear & Wrist PPG peaks 612 and 613 is about 32 msec. A PTT technique determines the time between a peak point, 612, to a base point, 611, in the ear PPG waveform is about 211 msec. The PTT in wrist PPG waveform 610-3 is about 243 msec. The blood pressure under the above conditions is: Sys:132 mmHg/Dia:88 mmHg/HR:75/minute. Accordingly, in some embodiments, the above features may be correlated to find, for example, a systolic blood pressure (SBP) as a function of PTT, or a PTT difference between features from ear and wrist PPG waveforms 610-1 and 610-3, or a time delay between ear and wrist PPG waveforms 610-1 and 610-3, or a time delay between features in ear PPG waveforms 610-1 and 610-3 and features in ECG waveform 610-2.

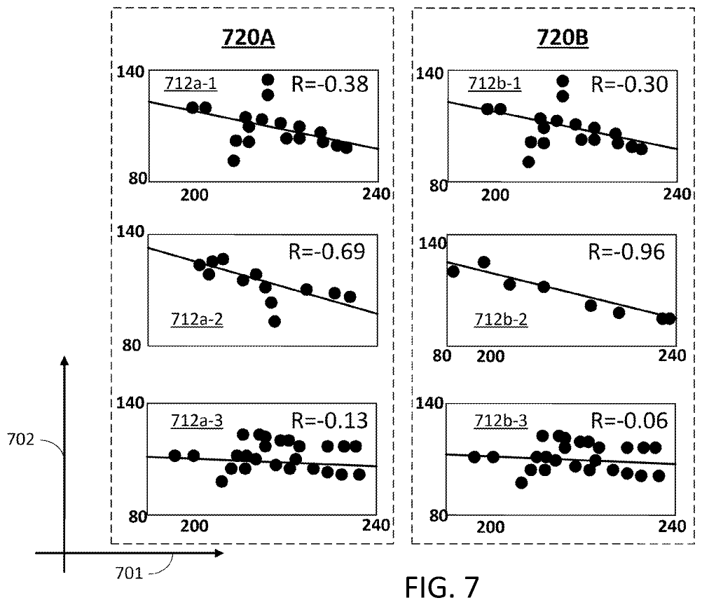

FIG. 7 illustrates regression methods 720A and 720B to combine features in waveforms 610 to identify a health condition of a user of an in-ear device, according to some embodiments. For example, different plots may be obtained for correlating PTT and SBP measurements 701 and 702, respectively, under different conditions: day (712a-1 and 712b-1), night (712a-2 and 712b-2), and full day cycle (712a-3 and 712b-3). Accordingly, a measurement of a PTT from one of an ear or wrist PPG waveform may provide an indication of an SBP. To enhance the accuracy of the SBP prediction, the user may be instructed to perform a one-time calibration using a ground-truth blood pressure system (e.g., a cuff-based arm blood pressure monitor). In such embodiment, the user may capture the ground-truth BP information while the sensor signals data are being captured using in-ear device, glasses, and/or wrist; or combination of all these. The user may need to capture the ground-truth BP information at rest and at an elevated blood pressure value (e.g., when the user is doing some exercise) to achieve such regression curve at a variety of SBP values.

In some embodiments, in-ear microphones and contact microphones may retrieve body-borne infrasound and low frequency sounds associated with a user's vital signs. Signal processing techniques, in conjunction with artificial intelligence (AI) processing, can be used to extract user's vital signs from these acoustic waveforms (e.g., heart rate, heart rate variability, breathing rate, and blood pressure).

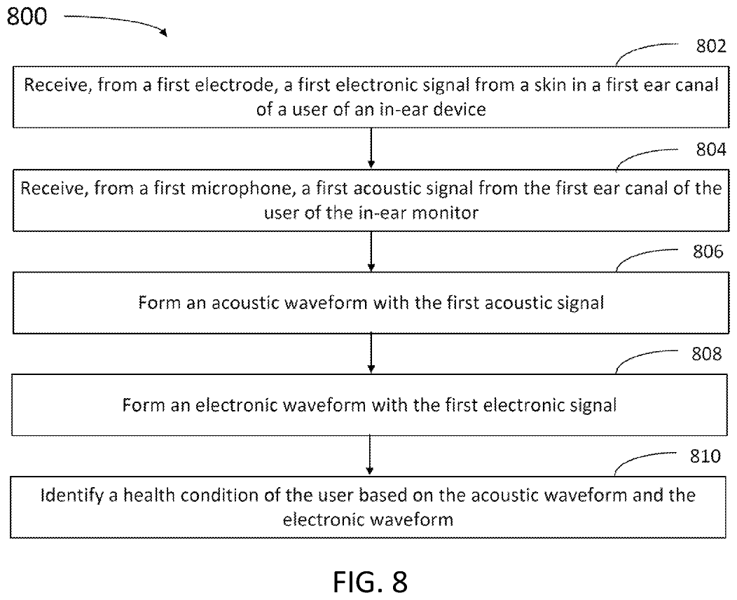

FIG. 8 is a flow chart illustrating steps in a method 800 for using microphones, motion sensors, optical sensors, electrodes, and a thermometer in an in-ear monitor for assessing the health of a user of a headset or smart glass, according to some embodiments. In some embodiments, at least one or more of the steps in method 800 may be performed by a processor executing instructions stored in a memory in either one of a smart glass or other wearable device on a user's body part (e.g., head, arm, wrist, leg, ankle, finger, toe, knee, shoulder, chest, back, and the like). In some embodiments, at least one or more of the steps in method 800 may be performed by a processor executing instructions stored in a memory, wherein either the processor or the memory, or both, are part of a mobile device for the user, a remote server or a database, communicatively coupled with each other via a network (cf., processors 112, 312, and memory 120, client devices 110, server 130, database 152, and network 150). Moreover, the mobile device, the smart glass, and the wearable devices may be communicatively coupled with each other via a wireless communication system and protocol (e.g., communications module 118, radio, Wi-Fi, Bluetooth, near-field communication—NFC—and the like). In some embodiments, a method consistent with the present disclosure may include one or more steps from method 800 performed in any order, simultaneously, quasi-simultaneously, or overlapping in time.

Step 802 includes receiving, from a first electrode, a first electronic signal from a skin in a first ear canal of a user of an in-ear device. In some embodiments, step 802 includes receiving, from a second electrode, a second electronic signal from the skin in the first ear canal of the user of the in-ear device and removing an interference from the first electronic signal with the first electronic signal. In some embodiments, step 802 includes receiving, from a second electrode, a second electronic signal from the skin in a second ear canal of the user of the in-ear device, and identifying an eye gaze direction based on the first electronic signal and the second electronic signal. In some embodiments, step 802 includes receiving an acoustic signal from an external microphone in the in-ear device in response to an acoustic stimulus; correlating the acoustic signal with the first electronic signal; and assessing a user response to the acoustic stimulus based on a brain activity from the first electronic signal and the acoustic stimulus. In some embodiments, step 802 includes receiving, from a second microphone, a second acoustic signal from the first ear canal of the user of the in-ear monitor; forming a second waveform with the first acoustic signal filtered from the second acoustic signal; and providing the second waveform to the user via a speaker, wherein the second acoustic signal is an audio signal from an external environment of the user. In some embodiments, step 802 includes providing, with a speaker, a sound signal into the first ear canal, for the user, wherein the first acoustic signal includes a back reflection of the sound signal, from an inner ear, and wherein identifying a health condition of the user includes determining a hearing condition of the user based on a delay and amplitude of the back reflection of the sound signal. In some embodiments, the first acoustic signal includes a sound gesture generated by the user as an input command, and step 802 includes identifying the input command from the acoustic waveform, and having a processor in a smart glass to execute the input command.

In some embodiments, to improve a signal to noise ratio, step 802 may include receiving, from a second electrode, a first electronic signal from a skin in a first ear canal of a user of an in-ear device binaural capture and processing (meaning that these data are simultaneously captured from both left and right ears).

Step 804 includes receiving, from a first microphone, a first acoustic signal from the first ear canal of the user of the in-ear monitor.

Step 806 includes forming an acoustic waveform with the first acoustic signal.

Step 808 includes forming an electronic waveform with the first electronic signal.

Step 810 includes identifying a health condition of the user based on the acoustic waveform and the electronic waveform. In some embodiments, step 810 includes performing a spectral analysis on the electronic waveform to identify a p-wave, a QRS-complex, and a T-wave complex in an electro-cardiogram. In some embodiments, the first acoustic signal is an internal signal from a body of the user and step 810 includes determining a heart rate of the user based on the acoustic waveform. In some embodiments, step 810 includes generating a spectrogram of the acoustic waveform; and identifying at least one of a heart rate value or a blood pressure value from the spectrogram of the acoustic waveform. In some embodiments, the first acoustic signal is an internal signal from a body of the user and step 810 includes determining a heart rate of the user. In some embodiments, step 810 includes identifying a systolic portion and a diastolic portion of the acoustic waveform based on a correlation of the first electronic signal with the first acoustic signal, and determining a blood pressure value with the systolic portion and the diastolic portion of the acoustic waveform. In some embodiments, step 810 includes: receiving, from a motion sensor in an in-ear device, a motion signal indicative of an inner body motion or a bulk body motion of a user of the in-ear device, and forming a motion waveform with the motion signal, wherein identifying a health condition of the user includes comparing the electronic waveform with the motion waveform. In some embodiments, step 810 includes receiving, from a second motion sensor in a second in-ear device, a second motion signal indicative of the inner body motion or the bulk body motion, and wherein forming the motion waveform includes removing at least one of a noise component or an interference from the motion signal with the second motion signal. In some embodiments, the motion signal is indicative of an inner body motion and step 810 includes determining at least one of a heart rate and a breathing rate of the user based on the inner body motion. In some embodiments, the motion signal is indicative of a bulk body motion of the user and step 810 includes at least one of detecting a fall of the user, a cough of the user, a sneeze of the user, or determining a step count for the user. In some embodiments, step 810 includes identifying a systolic heart pulse and a diastolic heart pulse from the motion waveform, and step 810 includes identifying features in the systolic heart pulse and the diastolic heart pulse. In some embodiments, step 810 includes filtering, with the motion waveform, one of a noise component and an interference from an audio signal to a speaker mounted in the in-ear device, and providing, with the speaker, the audio signal to the user. In some embodiments, step 810 includes receiving, from a temperature sensor, a temperature signal indicative of an inner body temperature of a user of an in-ear device, and forming a temperature waveform with the temperature signal, wherein identifying a health condition of the user includes comparing one of the electronic waveform or the acoustic waveform with the temperature waveform. In some embodiments, receiving a temperature signal includes collecting, with an infrared detector and a filter, an infrared radiation in a pre-selected bandwidth from an ear canal of the user. In some embodiments, receiving a temperature signal includes filtering an infrared radiation within a bandwidth based on a detection sensitivity over a bodily temperature range. In some embodiments, step 810 includes modeling a black body emitter having a selected emissivity of an ear canal of the user within a selected bandwidth, and determining the inner body temperature based on the selected emissivity of the ear canal. In some embodiments, receiving a temperature signal includes emitting an infrared radiation into an ear canal (or on the eardrum) of the user and determining an emissivity and absorbance of a tissue in the ear canal within a pre-selected bandwidth to calibrate the temperature signal. In some embodiments, receiving a temperature signal includes providing an infrared radiation to an in-ear canal (or eardrum) and collecting a backscattered infrared radiation to calibrate an emissivity value and absorbance value for the in-ear canal (or eardrum) into the temperature waveform. In some embodiments, receiving a temperature signal includes receiving a voltage value from a thermocouple electrode in contact with an in-ear canal of the user.

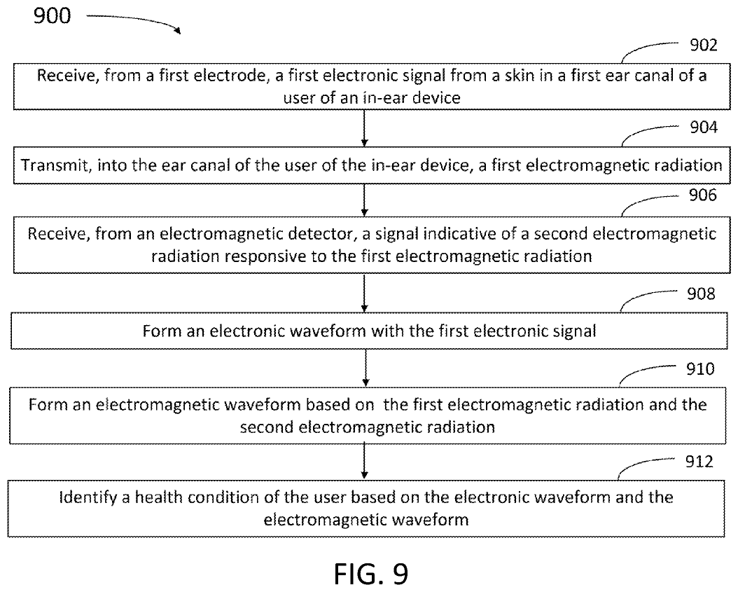

FIG. 9 is a flow chart illustrating steps in a method 900 for using microphones, motion sensors, optical sensors, electrodes, and a thermometer in an in-ear monitor for assessing the health of a user of a headset or smart glass, according to some embodiments. In some embodiments, at least one or more of the steps in method 900 may be performed by a processor executing instructions stored in a memory in either one of a smart glass or other wearable device on a user's body part (e.g., head, arm, wrist, leg, ankle, finger, toe, knee, shoulder, chest, back, and the like). In some embodiments, at least one or more of the steps in method 900 may be performed by a processor executing instructions stored in a memory, wherein either the processor or the memory, or both, are part of a mobile device for the user, a remote server or a database, communicatively coupled with each other via a network (cf., processors 112, 312, and memory 120, client devices 110, server 130, database 152, and network 150). Moreover, the mobile device, the smart glass, and the wearable devices may be communicatively coupled with each other via a wireless communication system and protocol (e.g., communications module 118, radio, Wi-Fi, Bluetooth, near-field communication—NFC—and the like). In some embodiments, a method consistent with the present disclosure may include one or more steps from method 900 performed in any order, simultaneously, quasi-simultaneously, or overlapping in time.

Step 902 includes receiving, from a first electrode, a first electronic signal from a skin in a first ear canal of a user of an in-ear device.

Step 904 includes transmitting, into the ear canal of the user of the in-ear device, a first electromagnetic radiation. In some embodiments, the first electromagnetic radiation includes a time-multiplex code and receiving a signal indicative of the second electromagnetic radiation includes decoding the signal according to the time-multiplex code. In some embodiments, step 904 may include transmitting multiple laser sources (e.g., each covering a number of different wavelength ranges from λ₁ to κₙ, covering a wide bandwidth from the near infrared to infrared (˜700 nm to 4μ wavelength range)). In some embodiments, step 904 includes sequentially illuminating the ear canal, each laser at a time.

Step 906 includes receiving, from an electromagnetic detector, a signal indicative of a second electromagnetic radiation responsive to the first electromagnetic radiation. In some embodiments, the second electromagnetic radiation is indicative of a change in an optical property of a functional layer in a chip embedded in the in-ear device, and step 906 includes determining a presence of a pre-selected target substance based on the change in the optical property of the functional layer, wherein the health condition is correlated with the presence of the pre-selected target substance. In some embodiments, the second electromagnetic radiation corresponds to the transmission and/or reflection of each of the multiple laser sources in the specific wavelength range, and is measured using the detector.

Step 908 includes forming an electronic waveform with the first electronic signal.

Step 910 includes forming an electromagnetic waveform based on the first electromagnetic radiation and the second electromagnetic radiation.

Step 912 includes identifying a health condition of the user based on the electronic waveform and the electromagnetic waveform. In some embodiments, the first electromagnetic radiation is in resonance with a plasmon mode of a metallic layer disposed in the in-ear device, and step 912 includes determining a presence of a pre-selected target substance based on a change of plasmon resonance to the second electromagnetic radiation. In some embodiments, the second electromagnetic radiation includes a backscattered portion of the first electromagnetic radiation and step 912 includes identifying a cardio-respiratory condition based on a waveform of the backscattered portion of the first electromagnetic radiation. In some embodiments, a difference between the first electromagnetic radiation and the second electromagnetic radiation is indicative of a trace amount of a selected molecule in the ear canal of the user and identifying health condition of the user includes determining that a concentration of the selected molecule is higher than a healthy threshold value. In some embodiments, the health condition of the user is a diabetic condition, and step 912 includes monitoring a blood-glucose level for the user. Accordingly, the second electromagnetic radiation may indicate a unique reflection and transmission spectrum indicative of a blood-glucose level of the user. Thus, step 912 may include identifying multi-band reflection spectra from blood vessels of the in-ear canal for remotely and continuously monitoring blood-glucose levels for the user.

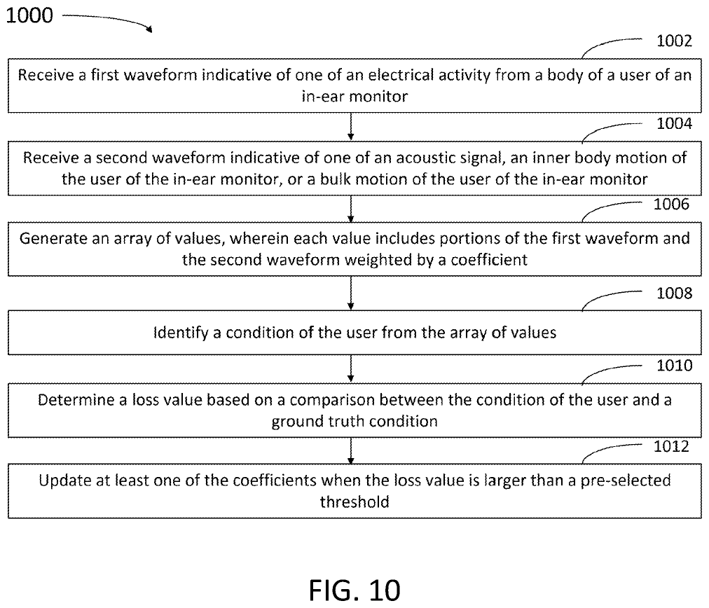

FIG. 10 is a flow chart illustrating steps in a method 1000 for using microphones, motion sensors, optical sensors, electrodes, and a thermometer in an in-ear monitor for assessing the health of a user of a headset or smart glass, according to some embodiments. In some embodiments, at least one or more of the steps in method 1000 may be performed by a processor executing instructions stored in a memory in either one of a smart glass or other wearable device on a user's body part (e.g., head, arm, wrist, leg, ankle, finger, toe, knee, shoulder, chest, back, and the like). In some embodiments, at least one or more of the steps in method 1000 may be performed by a processor executing instructions stored in a memory, wherein either the processor or the memory, or both, are part of a mobile device for the user, a remote server or a database, communicatively coupled with each other via a network (cf., processors 112, 312, and memory 120, client devices 110, server 130, database 152, and network 150). Moreover, the mobile device, the smart glass, and the wearable devices may be communicatively coupled with each other via a wireless communication system and protocol (e.g., communications module 118, radio, Wi-Fi, Bluetooth, near-field communication—NFC—and the like). In some embodiments, a method consistent with the present disclosure may include one or more steps from method 1000 performed in any order, simultaneously, quasi-simultaneously, or overlapping in time.

Step 1002 includes receiving a first waveform indicative of one of an electrical activity from a body of a user of an in-ear monitor.

Step 1004 includes receiving a second waveform indicative of one of an acoustic signal, an inner motion of the user of the in-ear monitor, or a bulk motion of the user of the in-ear monitor.

Step 1006 includes generating an array of values, wherein each value includes portions of the first waveform and the second waveform weighted by a coefficient. In some embodiments, step 1006 includes forming a two-dimensional convolution of each of the first waveform and the second waveform with a baseline waveform retrieved for the user of the in-ear monitor from a history database. In some embodiments, step 1006 includes retrieving a baseline waveform for multiple users from a healthcare database and convolving the first waveform and the second waveform with the baseline waveform. In some embodiments, the first waveform is an electro-encephalogram, and the second waveform is a sound waveform, and step 1006 includes correlating the electro-encephalogram with the sound waveform to generate a value for an attention coefficient of the user to a source of the sound waveform. In some embodiments, step 1006 includes associating a classifier to a portion of the first waveform or the second waveform, the classifier associated with a vital sign from the user. In some embodiments, the first waveform is an electro-cardiogram, and the second waveform is indicative of a cardiovascular motion in the body of the user, and step 1006 includes correlating the electro-cardiogram with the cardiovascular motion in the body of the user, and the array of values including at least one feature indicative of a blood pressure of the user. In some embodiments, the inner motion of the user is a sneeze or a cough, the second waveform is indicative of an inner body temperature of the user, and step 1006 includes correlating the inner body temperature with an inner motion of the user and a disease condition. In some embodiments, the inner motion of the user is a sneeze or a cough, the second waveform is indicative of an inner body temperature of the user, and step 1006 includes correlating the inner body temperature with an inner motion of the user and a disease condition. In some embodiments, the first waveform is an electro-encephalogram, and step 1006 includes determining a correlation value between the acoustic signal and the electro-encephalogram, and identifying the condition of the user includes identifying an auditive response threshold based on an amplitude of the acoustic signal and the correlation value.

Step 1008 includes identifying a condition of the user from the array of values. In some embodiments, the first waveform is indicative of a position of an eye of the user, and step 1008 includes determining a gaze direction of the user.

Step 1010 includes determining a loss value based on a comparison between the condition of the user and a ground truth condition.

Step 1012 includes updating at least one of the coefficients when the loss value is larger than a pre-selected threshold.

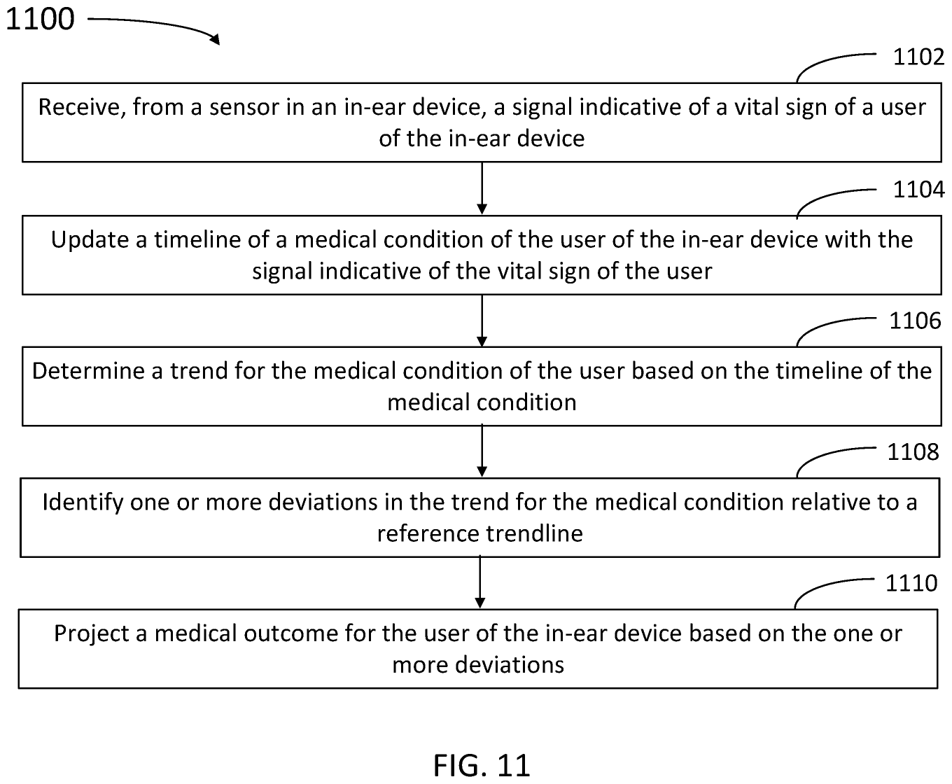

FIG. 11 is a flow chart illustrating steps in a method 1100 for creating a data of measurements over time to create a reference and trendline, according to some embodiments. In some embodiments, at least one or more of the steps in method 1100 may be performed by a processor executing instructions stored in a memory in either one of a smart glass or other wearable device on a user's body part (e.g., head, arm, wrist, leg, ankle, finger, toe, knee, shoulder, chest, back, and the like). In some embodiments, at least one or more of the steps in method 1000 may be performed by a processor executing instructions stored in a memory, wherein either the processor or the memory, or both, are part of a mobile device for the user, a remote server or a database, communicatively coupled with each other via a network (cf., processors 112, 312, and memory 120, client devices 110, server 130, database 152, and network 150). Moreover, the mobile device, the smart glass, and the wearable devices may be communicatively coupled with each other via a wireless communication system and protocol (e.g., communications module 118, radio, Wi-Fi, Bluetooth, near-field communication—NFC—and the like). In some embodiments, a method consistent with the present disclosure may include one or more steps from method 1100 performed in any order, simultaneously, quasi-simultaneously, or overlapping in time.

Step 1102 includes receiving, from a sensor in an in-ear device, a signal indicative of a vital sign of a user of the in-ear device. In some embodiments, step 1102 includes combining multiple signals from one or more sensors in the in-ear device, the signals comprising at least an electronic signal, an acoustic signal, and an optical signal. In some embodiments, step 1102 includes receiving an electrocardiogram signal and an acoustic signal, and updating a timeline of a medical information comprises combining the electrocardiogram signal and the acoustic signal to determine a blood pressure value for the user of the in-ear device. In some embodiments, step 1102 includes receiving, from a mobile device communicatively coupled with the in-ear device, a command to adjust a configuration setting for the sensor in the in-ear device based on the one or more deviations in the trend.

Step 1104 includes updating a timeline of a medical condition of the user of the in-ear device with the signal indicative of the vital sign from the user. In some embodiments, the in-ear device is communicatively coupled with an immersive reality headset worn by the user of the in-ear device, and step 1104 includes transmitting, from the immersive reality headset to a remote server, the timeline of the medical condition for the user of the in-ear device.

Step 1106 includes determining a trend for the medical condition of the user based on the timeline of the medical condition. In some embodiments, the in-ear device is communicatively coupled with a mobile device for the user of the in-ear device, and step 1106 includes accessing an application in the mobile device storing multiple prior values indicative of the vital sign from the user of the in-ear device.

Step 1108 includes identifying one or more deviations in the trend for the medical condition relative to a reference trendline. In some embodiments, step 1108 includes receiving, from a remote database, the reference trendline. In some embodiments, step 1108 includes forming the reference trendline based on multiple timelines for medical conditions of a population of individuals having a demographic profile of the user of the in-ear device.

Step 1110 includes projecting a medical outcome for the user of the in-ear device based on the one or more deviations. In some embodiments, step 1110 includes receiving, form a remote server, the medical outcome for the user of the in-ear device. In some embodiments, step 1110 includes anonymizing the trend for the medical condition of the user and transmitting the trend of the medical condition of the user to a remote server communicatively coupled with the in-ear device.

### Hardware Overview

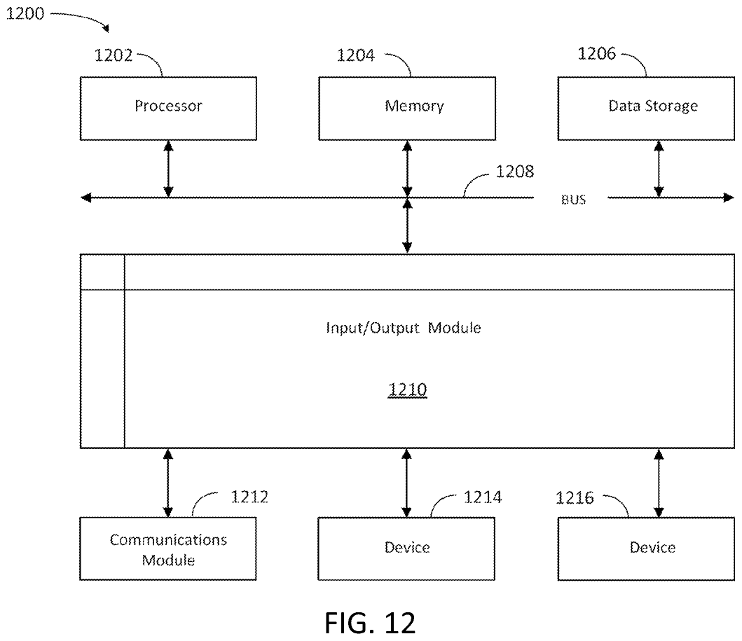

FIG. 12 is a block diagram illustrating an exemplary computer system 1200 with which headsets and other client devices 110, and methods 800-1100 can be implemented, according to some embodiments. In certain aspects, computer system 1200 may be implemented using hardware or a combination of software and hardware, either in a dedicated server, or integrated into another entity, or distributed across multiple entities. Computer system 1200 may include a desktop computer, a laptop computer, a tablet, a phablet, a smartphone, a feature phone, a server computer, or otherwise. A server computer may be located remotely in a data center or be stored locally.

Computer system 1200 includes a bus 1208 or other communication mechanism for communicating information, and a processor 1202 (e.g., processors 112) coupled with bus 1208 for processing information. By way of example, the computer system 1200 may be implemented with one or more processors 1202. Processor 1202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1200 can include, in addition to hardware, code that creates an execution environment for the computer program in question, e.g., code that constitutes processor firmware, a protocol stack, a database management system, an operating system, or a combination of one or more of them stored in an included memory 1204 (e.g., memory 120), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled with bus 1208 for storing information and instructions to be executed by processor 1202. The processor 1202 and the memory 1204 can be supplemented by, or incorporated in, special purpose logic circuitry.

The instructions may be stored in the memory 1204 and implemented in one or more computer program products, e.g., one or more modules of computer program instructions encoded on a computer-readable medium for execution by, or to control the operation of, the computer system 1200, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1204 may also be used for storing temporary variable or other intermediate information during execution of instructions to be executed by processor 1202.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 1200 further includes a data storage device 1206 such as a magnetic disk or optical disk, coupled with bus 1208 for storing information and instructions. Computer system 1200 may be coupled via input/output module 1210 to various devices. Input/output module 1210 can be any input/output module. Exemplary input/output modules 1210 include data ports such as USB ports. The input/output module 1210 is configured to connect to a communications module 1212. Exemplary communications modules 1212 include networking interface cards, such as Ethernet cards and modems. In certain aspects, input/output module 1210 is configured to connect to a plurality of devices, such as an input device 1214 and/or an output device 1216. Exemplary input devices 1214 include a keyboard and a pointing device, e.g., a mouse or a trackball, by which a consumer can provide input to the computer system 1200. Other kinds of input devices 1214 can be used to provide for interaction with a consumer as well, such as a tactile input device, visual input device, audio input device, or brain-computer interface device. For example, feedback provided to the consumer can be any form of sensory feedback, e.g., visual feedback, auditory feedback, or tactile feedback; and input from the consumer can be received in any form, including acoustic, speech, tactile, or brain wave input. Exemplary output devices 1216 include display devices, such as an LCD (liquid crystal display) monitor, for displaying information to the consumer.

According to one aspect of the present disclosure, headsets and client devices 110 can be implemented, at least partially, using a computer system 1200 in response to processor 1202 executing one or more sequences of one or more instructions contained in memory 1204. Such instructions may be read into memory 1204 from another machine-readable medium, such as data storage device 1206. Execution of the sequences of instructions contained in main memory 1204 causes processor 1202 to perform the process steps described herein. One or more processors in a multi-processing arrangement may also be employed to execute the sequences of instructions contained in memory 1204. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data server, or that includes a middleware component, e.g., an application server, or that includes a front end component, e.g., a client computer having a graphical consumer interface or a Web browser through which a consumer can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1200 can include clients and servers. A client and server are generally remote from each other and typically interact through a communication network. The relationship of client and server arises by virtue of computer programs running on the respective computers and having a client-server relationship to each other. Computer system 1200 can be, for example, and without limitation, a desktop computer, laptop computer, or tablet computer. Computer system 1200 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 1202 for execution. Such a medium may take many forms, including, but not limited to, non-volatile media, volatile media, and transmission media. Non-volatile media include, for example, optical or magnetic disks, such as data storage device 1206. Volatile media include dynamic memory, such as memory 1204. Transmission media include coaxial cables, copper wire, and fiber optics, including the wires forming bus 1208. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public, regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A computer-implemented method, comprising:
receiving, from a first electrode, a first electronic signal from a skin in a first ear canal of a user of an in-ear device;
receiving, from a first microphone, a first acoustic signal from the first ear canal of the user of an in-ear monitor;
forming an acoustic waveform with the first acoustic signal;
forming an electronic waveform with the first electronic signal; and
identifying a health condition of the user based on the acoustic waveform and the electronic waveform.

2. The computer-implemented method of claim 1, further comprising receiving, from a second electrode, a second electronic signal from the skin in the first ear canal of the user of the in-ear device, and removing an interference from the first electronic signal with the first electronic signal.

3. The computer-implemented method of claim 1, further comprising receiving, from a second electrode, a second electronic signal from the skin in a second ear canal of the user of the in-ear device, and identifying an eye gaze direction based on the first electronic signal and the second electronic signal.

4. The computer-implemented method of claim 1, further comprising receiving an acoustic signal from an external microphone in the in-ear device in response to an acoustic stimulus; correlating the acoustic signal with the first electronic signal; and assessing a user response to the acoustic stimulus based on a brain activity from the first electronic signal and the acoustic stimulus.

5. The computer-implemented method of claim 1, further comprising receiving, from a second microphone, a second acoustic signal from the first ear canal of the user of the in-ear monitor; forming a second waveform with the first acoustic signal filtered from the second acoustic signal; and providing the second waveform to the user via a speaker, wherein the second acoustic signal is an audio signal from an external environment of the user.

6. The computer-implemented method of claim 1, further comprising providing, with a speaker, a sound signal into the first ear canal, for the user, wherein the first acoustic signal comprises a back reflection of the sound signal, from an inner ear, and wherein identifying a health condition of the user comprises determining a hearing condition of the user based on a delay and amplitude of the back reflection of the sound signal.

7. The computer-implemented method of claim 1, wherein the first acoustic signal includes a sound gesture generated by the user as an input command, further comprising identifying the input command from the acoustic waveform, and having a processor in a smart glass to execute the input command.

8. The computer-implemented method of claim 1, wherein identifying a health condition of the user further comprises performing a spectral analysis on the electronic waveform to identify a p-wave, a QRS-complex, and a T-wave complex in an electro-cardiogram.

9. The computer-implemented method of claim 1, wherein the first acoustic signal is an internal signal from a body of the user and identifying a health condition of the user comprises determining a heart rate of the user based on the acoustic waveform.

10. The computer-implemented method of claim 1, wherein identifying a health condition of the user comprises generating a spectrogram of the acoustic waveform; and identifying at least one of a heart rate value or a blood pressure value from the spectrogram of the acoustic waveform.

11. A computer-implemented method, comprising:
receiving, from a sensor in an in-ear device, a signal indicative of a vital sign of a user of the in-ear device;
updating a timeline of a medical condition of the user of the in-ear device with the signal indicative of the vital sign from the user;
determining a trend for the medical condition of the user based on the timeline of the medical condition;
identifying one or more deviations in the trend for the medical condition relative to a reference trendline; and
projecting a medical outcome for the user of the in-ear device based on the one or more deviations.

12. The computer-implemented method of claim 11, wherein receiving a signal indicative of a vital sign of a user of the in-ear device comprises combining multiple signals from one or more sensors in the in-ear device, the signals comprising at least an electronic signal, an acoustic signal, and an optical signal.

13. The computer-implemented method of claim 11, wherein receiving a signal indicative of a vital sign of a user of the in-ear device comprises receiving an electrocardiogram signal and an acoustic signal, and updating a timeline of a medical information comprises combining the electrocardiogram signal and the acoustic signal to determine a blood pressure value for the user of the in-ear device.

14. The computer-implemented method of claim 11, wherein the in-ear device is communicatively coupled with an immersive reality headset worn by the user of the in-ear device, further comprising transmitting, from the immersive reality headset to a remote server, the timeline of the medical condition for the user of the in-ear device.

15. The computer-implemented method of claim 11, wherein the in-ear device is communicatively coupled with a mobile device for the user of the in-ear device, and determining a trend for the medical condition based on the timeline comprises accessing an application in the mobile device storing multiple prior values indicative of the vital sign from the user of the in-ear device.

16. The computer-implemented method of claim 11, further comprising receiving, from a mobile device communicatively coupled with the in-ear device, a command to adjust a configuration setting for the sensor in the in-ear device based on the one or more deviations in the trend.

17. The computer-implemented method of claim 11, further comprising receiving, from a remote database, the reference trendline.

18. The computer-implemented method of claim 11, wherein projecting a medical outcome for the user of the in-ear device comprises receiving, form a remote server, the medical outcome for the user of the in-ear device.

19. The computer-implemented method of claim 11, further comprising forming the reference trendline based on multiple timelines for medical conditions of a population of individuals having a demographic profile of the user of the in-ear device.

20. The computer-implemented method of claim 11, further comprising anonymizing the trend for the medical condition of the user and transmitting the trend of the medical condition of the user to a remote server communicatively coupled with the in-ear device.
